(19) United States
(12) Patent Application Publication
(10) Pub. No.: US 2020/0179190 A1
(43) Pub. Date: Jun. 11, 2020

(54) SYSTEM HAVING AN ABSORBENT ARTICLE WITH SEPARATE FASTENER

(71) Applicant: Kimberly-Clark Worldwide, Inc., Neenah, WI (US)

(72) Inventors: Angela A. JOHNSTON, New London, WI (US); Jackalyn M. WILLIAMS, Menasha, WI (US); Barbara J. VOIGTMAN, Greenville, WI (US)

(21) Appl. No.: 16/608,578

(22) PCT Filed: Apr. 28, 2017
(86) PCT No.: PCT/US17/30019

§ 371 Date: Oct. 25, 2019

(51) Int. Cl. A61F 13/56

(52) U.S. Cl. CPC A61F 13/5638; A61F 2013/15008

(57) ABSTRACT

The present invention relates to a system for providing skin protection for premature infants where the system includes a rectangular absorbent article and a detached fastener. The rectangular absorbent article includes an outer layer, an inner, skin facing layer and an absorbent structure located between the outer layer and the inner, skin facing layer. The length of the rectangular absorbent article is less than 310 mm and the width of the rectangular absorbent article is less than 200 mm. The detached fastener has two, lateral end portions; each of the lateral end portions includes an engagement material. The length of the engagement material is less than 35 mm and the width of the engagement material is less than 45 mm.

### BACKGROUND

A primary function of personal care absorbent articles is to absorb and retain body exudates such as urine, fecal material, blood, and menses with additional desired attributes including low leakage of the exudates from the absorbent article and a dry feel to the wearer of the absorbent article. To accomplish these tasks, personal care absorbent articles generally have an absorbent core and a cover enclosing the absorbent core. The cover is usually fluid pervious on the body facing side of the absorbent core and fluid impervious on the garment facing side of the absorbent core. While the usage of absorbent articles is well-known, the design and function of such articles can be significant when the articles are used in the care of infants who are born prematurely. Several manufacturers offer diapers that are intended to be used with “Preemies” (babies weighing less than 6 lbs. at birth); “MicroPreemies” (babies weighing less than 4 lbs. at birth); and “NanoPreemies” (babies weighing less than 2 lbs. at birth).

It is well-known that the skin of premature infants is under-developed. Premature infant skin has a thinner epidermis compared to full-term infant skin (up to half as thick with very premature infants). Consequently, premature skin has less physical barrier protection. Premature infant skin is also very permeable to both water and irritants; this is caused by an under-developed stratum corneum compared to full-term infant skin. Further, premature infant skin has less structural proteins in the dermal layer, rendering a weaker junction between the dermis and epidermis compared to full-term infant skin. This results in skin being more easily damaged by mechanical action such as cleansing of skin for removal of urine or feces. In addition to their skin being under-developed, premature infants may also have medical challenges for which they require care and treatment. Depending on the magnitude of prematurity, premature infants typically spend extended periods of time in hospital care (such as in a neonatal intensive care unit “NICU”). The devices and equipment needed to deliver care and treatment needs to be integrated with more fundamental articles, such as absorbent articles (that is, diapers). Ideally, all of the lines and tubes are integrated and operate harmoniously with each other, but this state in which placement of lines and tubes is maintained (despite the infant's movement and the interventions of caregivers) is not always achieved.

For example, if an infant has an abdominal wound, intravenous/arterial line (umbilical catheter) or a lower back issue such as a Myelomeningocele (when part of the spinal cord and nerves are outside the body) on the lower spine with spina bifida, a nurse will need to adjust how the infant's diaper (the absorbent article) fits. Absorbent articles having attached fasteners can only land in a limited number of locations to accommodate the challenges of a wound or an IV line. Because of the situations noted where a conventional diaper may not fit due to the location of the fixed fasteners, the infant may not have a diaper placed—or, if the diaper is placed, it may not be secured—leading to possible contamination of the line or wound/open area from urine or stool. The nurse may also pad the diaper with other absorbent material if the diaper is not secured in the optimal manner because of the anatomical obstacles previously described (line/wound/myelomeningocele). The use of these additional absorbents (such as cotton balls or gauze) may not contain the urine or stool and may lead to skin irritation if not changed when wet/soiled since the body fluid will not be wicked away as it would with superabsorbent material (“SAM”).

In other instances, the infant may have abdominal distention related to mechanical ventilation or feeding intolerance/constipation or even NEC (Necrotizing enterocolitis: a serious disease that occurs when the intestinal tissue becomes damaged and begins to die; it most often affects premature infants and common symptoms include bloating or swelling in the abdomen, bloody stools, and diarrhea). As a result of their distended abdomen, the nurse may not be able to close the diaper and will need to either go up in a diaper size to accommodate the waist or not fasten the diaper; either of these options can lead to increased risk of leakage of the diaper. Leakage of the diaper requires a bed change which with premature infants can be a huge task if the infant is critically ill with many intravenous lines, mechanical ventilation, drainage tubes, etc. For critically ill infants, a bed change can require multiple staff and increases the risk of dislodging the life sustaining lines/tubes the infant has.

Another intervention that premature infants often require is to be connected to an EKG. In practice, nurses commonly use the attached fastener of conventional diapers to secure leads from the EKG. This works to help keep the leads from getting entangled with other equipment, but is not optimal because the fasteners are only located in one position on the diaper. Depending on the infant's needs and additional equipment, it may be better for the infant to secure the leads at another location on the diaper. This is not possible with diapers having attached fasteners.

In the NICU, nurses may use diapers for other purposes. For example, because of its insulating and absorptive features, a diaper may be used to warm an infant's foot for a heel stick or to measure/prevent leaks from feeding/decompression tubes or ostomy collection devices. For diapers with attached fasteners, the attached fasteners may not allow for complete fitting around the infant's foot or around the feeding/decompression tubes or ostomy collection devices. The result for suboptimal warming may be that the infant's foot is not be warmed enough to allow free flow of blood with blood collection. This could lead to increased infant discomfort and skewed lab values (i.e. falsely increased potassium levels due to cell trauma with squeezing the foot too much/too tightly to get blood) or in the case of body fluid collection, it could lead to leaks and inaccurate output measures. With conventional diapers having attached fasteners, the diapers can easily be secured with an infant lying on their back. Conventional diapers can also work being applied with the infant on their abdomen. However, if the infant is in any other position, application of a conventional diaper with attached fasteners will most likely require that the infant would need to be moved. Repeated, unnecessary movement of the sleeping infant can lead to long term issues. Further, some infants in the NICU require phototherapy and need to have as much skin exposed as possible. There is always the challenge of what to do with the diapered areas. Nurses may try to use smaller diapers to allow for more skin to be open to the phototherapy. This is not optimal as this can lead to leaks and an uncomfortable fit for the infant.

There remains a need for an absorbent article that has greater flexibility in placement and application, particularly for medically-complicated and/or premature infants being treated in a NICU. There also remains a need for an absorbent article having fasteners that are not fixed to a single location and a single orientation. Further, there remains a need for a system that provides greater functionality to absorbent articles to increase their effectiveness for all of the applications in which they are used.

### SUMMARY

The present invention relates generally to systems for providing skin protection for premature infants. The skin protection is provided by absorbing and separating body exudates from the infant's skin; skin protection is further provided by a combination of system elements that improve the positioning of the rectangular absorbent article around the infant's torso. In addition to providing skin protection, the system of the invention protects the infant by enabling secure placement of medical lines being used to support and treat the infant.

The system of the invention includes a rectangular absorbent article having a longitudinal direction and a lateral direction. The rectangular absorbent article has a length in the longitudinal direction and a width in the lateral direction. The rectangular absorbent article includes an outer layer, an inner, skin facing layer and an absorbent structure located between the outer layer and the skin facing layer. The system is designed for use with infants born before they are full term and, therefore, infants who are premature. The length of the rectangular absorbent article is less than 310 mm and the width of the rectangular absorbent article is less than 200 mm. The rectangular absorbent article is primarily rectangular in configuration with primary dimensions in a two-dimensional plane; however, the edges of the rectangular absorbent article may have a subtle contour or curve to them. The system also includes a detached fastener; the detached fastener has a length that is aligned or oriented with the lateral direction of the rectangular absorbent article. The detached fastener also has a width aligned or oriented with the longitudinal direction of the rectangular absorbent article. The detached fastener has two, lateral end portions. Each lateral end portion includes an engagement material. The engagement material defines a length and a width that are oriented with the length and the width of the detached fastener. The length of the engagement material on the detached fastener is less than 35 mm and the width of the engagement material on the detached fastener is less than 45 mm. Because of the size of the infants with which the system of the invention may be used, in one aspect, the width of the rectangular absorbent article is less than 150 mm. A width of this dimension addresses the smaller distance between the legs of a premature infant.

The detached fastener of the system of the invention allows greater flexibility in use for both securing the rectangular absorbent article and other objects in the infant's immediate environment. Therefore, in one aspect, the length of the detached fastener is at least 50 mm. The capability of the system of the invention may be expanded by inclusion of more than one detached fastener. The lateral end portions of the detached fastener (which both include an engagement material) may be connected to each other by a nonwoven material. The detached fasteners of the system may be formed entirely of a nonwoven material (such as a spunbond/meltblown/spunbond material) that has an engagement material integrated at the lateral end portions. Further, the nonwoven material may be an elastic material (for example, a necked-bonded laminate material). The engagement material on the lateral end portions may be a mechanical fastener, such as a hook material, or other suitable material. The engagement material may be selected to readily engage with the outer layer of the rectangular absorbent article. Further, the engagement material may be selected to readily engage, but also to readily be re-positioned without the system becoming unusable. The engagement material should be secure once placed, but should also be capable of being reasonably removed, too. This aspect is consistent with the intended use and benefits of the system.

In another aspect, the system of the invention includes a rectangular absorbent article having a longitudinal direction and a lateral direction. The rectangular absorbent article has a length in the longitudinal direction and a width in the lateral direction. The rectangular absorbent article includes an outer layer, an inner, skin facing layer and an absorbent structure located between the outer layer and the skin facing layer. The rectangular absorbent article is primarily rectangular in configuration with primary dimensions in a two-dimensional plane; however, the edges of the rectangular absorbent article may have a subtle contour or curve to them. The system also includes a detached fastener; the detached fastener has a length that is aligned or oriented with the lateral direction of the rectangular absorbent article. The detached fastener also has a width aligned or oriented with the longitudinal direction of the rectangular absorbent article. The detached fastener has two, lateral end portions. Each lateral end portion includes an engagement material. The engagement material defines a length and a width that are oriented with the length and the width of the detached fastener. In order to more specifically describe the dimensional relationship between the size of the rectangular absorbent article and the size of the detached fastener, the system may have a ratio of the length of the rectangular absorbent article to the width of the engagement material of the detached fastener of at least 20 to 3. In a further aspect, the system may have a ratio of the length of the rectangular absorbent article to the width of the engagement material of the detached fastener of less than 40 to 3. An appropriate length for the rectangular absorbent article to have is at least 180 mm. Because of the size of the infants with which the system of the invention may be used, in one aspect, the width of the rectangular absorbent article is less than 150 mm.

With this aspect of the invention, the length of the detached fastener may be at least 50 mm. The capability of the system of the invention may be expanded by inclusion of more than one detached fastener. The lateral end portions of the detached fastener (which both include an engagement material) may be connected to each other by a nonwoven material. The detached fasteners of the system may be formed entirely of a nonwoven material (such as a spunbond/meltblown/spunbond material) that has an engagement material integrated at the lateral end portions. Further, the nonwoven material may be an elastic material (for example, a necked-bonded laminate material). The engagement material on the lateral end portions may be a mechanical fastener, such as a hook material, or other suitable material. The dimensions of the detached fastener may be selected for optimal placement and handling by caregivers. When the width of the engagement material of the detached fastener is too great in relation to the length of the rectangular absorbent article, the engagement material may come into contact with the infant's skin. Taking this into consideration, the width of the engagement material may be less than 45 mm. However, the width of the engagement material needs to be sufficient to perform its intended function.

The present invention, including the exemplary embodiments, are described in greater detail in the Detailed Description along with the Drawings.

### BRIEF DESCRIPTION OF THE DRAWINGS

FIG. 1 is a top down view of an embodiment of an absorbent article of the invention.

FIG. 2 is a top down view of an embodiment of a detached fastener of the invention that—in combination with the absorbent article shown in FIG. 1—forms the system of the invention. The detached fastener is shown in both a “folded” and “unfolded” configuration. The “folded” configuration represents the appearance of the detached fastener prior to use and the “unfolded” configuration represents the appearance of the detached fastener when being put into use.

FIG. 3 is a top down view of a representative detached fastener of the system of the invention.

FIG. 4 is a front view of an example of the system of the invention. The front view is a view of how the system would appear when in use on an infant looking at the infant's chest and stomach area. The front view shows how the detached fasteners of the system may overlap each other in the front of the absorbent article in use.

FIG. 5 is side view of the system of the invention shown in FIG. 4. The side view is a view of how the system would appear when in use on an infant looking at the infant's hips. The detached fasteners are overlapping each other in this view.

FIG. 6 is a back view of the system of the invention shown in FIGS. 4 and 5. The back view is a view of how the system would appear when in use on an infant looking at the infant's back. The back view also shows how the detached fasteners of the system may overlap each other in the back of the absorbent article in use.

FIG. 7 is a front view of an example of the system of the invention in use. The view shows the absorbent article and a detached fastener that has been positioned on the outer cover of the absorbent article in such a way so as to securely place and hold a medical line, such as an EKG lead or a feeding tube.

### DETAILED DESCRIPTION

The present invention relates generally to a system for providing skin protection for premature infants. The system of the invention includes a rectangular absorbent article 10 having a longitudinal direction 30 and a lateral direction 32. The rectangular absorbent article 10 is generally rectangular in shape—meaning that it has one dimension that is longer than the other dimension in a two-dimensional plane; however, the edges of the rectangular absorbent article 10 do not need to be perfectly straight. The rectangular absorbent article 10 has a length in the longitudinal direction 30 and a width in the lateral direction 32. Because the system of the invention is generally intended for infants born prematurely, the rectangular absorbent article 10 may not have all of the features associated with conventional absorbent articles. However, the rectangular absorbent article 10 includes an outer layer 26, an inner, skin facing layer 28 and absorbent structure 40 between the outer layer 26 and the skin facing layer 28. As will be described in more detail herein, the rectangular absorbent article 10 will have specific dimensions identified to support the care of premature infants. The system of the invention also includes a detached fastener 140; the system of the invention may include more than one detached fastener 140, too. The detached fastener 140 has a length 142 aligned with the lateral direction 32 of the rectangular absorbent article 10 and a width 144 aligned with the longitudinal direction 30 of the rectangular absorbent article 10. The detached fastener 140 also includes two, lateral end portions 150. Each lateral end portion includes an engagement material 154. The system of the invention provides a previously unrecognized solution of providing a rectangular absorbent article 10 having dimensions that correspond to particular dimensions of engagement material 154 on a detached fastener 140. The system of the invention is particularly advantageous for providing care to premature infants.

In order to facilitate understanding of the present invention, the following is a listing of elements and their reference numerals as they are shown in the Figures.

| Reference Numeral | Element Name |
|---|---|
| 10 | Rectangular Absorbent Article |
| 12 | Length in the Longitudinal Direction |
| 14 | Width in the Lateral Direction |
| 20 | Longitudinal Side Edge |
| 22 | Waist Edge |
| 26 | Outer Layer |
| 28 | Skin Facing Layer |
| 30 | Longitudinal Direction |
| 32 | Lateral Direction |
| 40 | Absorbent Structure |
| 50 | Containment Flap |
| 66 | Leg Elastic Member |
| 140 | Detached Fastener |
| 142 | Length of Detached Fastener |
| 144 | Width of Detached Fastener |
| 146 | Length of Engagement Material |
| 148 | Width of Engagement Material |
| 150 | Lateral End Portion |
| 152 | Nonwoven Material |
| 154 | Engagement Material |
| 156 | Medical Line |

### Definitions

The term “absorbent article’ refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “fluid entangling” and “fluid entangled” refers herein to a formation process for further increasing the degree of fiber entanglement within a given fibrous nonwoven web or between fibrous nonwoven webs and other materials so as to make the separation of the individual fibers and/or the layers more difficult as a result of the entanglement. Generally this is accomplished by supporting the fibrous nonwoven web on some type of forming or carrier surface which has at least some degree of permeability to the impinging pressurized fluid. A pressurized fluid stream (usually multiple streams) can then be directed against the surface of the nonwoven web which is opposite the supported surface of the web. The pressurized fluid contacts the fibers and forces portions of the fibers in the direction of the fluid flow thus displacing all or a portion of a plurality of the fibers towards the supported surface of the web. The result is a further entanglement of the fibers in what can be termed the Z-direction of the web (its thickness) relative to its more planar dimension, its X-Y plane. When two or more separate webs or other layers are placed adjacent one another on the forming/carrier surface and subjected to the pressurized fluid, the generally desired result is that some of the fibers of at least one of the webs are forced into the adjacent web or layer thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, the fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling which is a well-known process to those of ordinary skill in the art of nonwoven webs. Examples of fluid entangling process can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

The term “g/cc” refers herein to grams per cubic centimeter.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The rectangular absorbent article 10 will be described in detail herein followed by a detailed description of the detached fastener 140—which, when used together, form the system of the invention.

### Rectangular Absorbent Article:

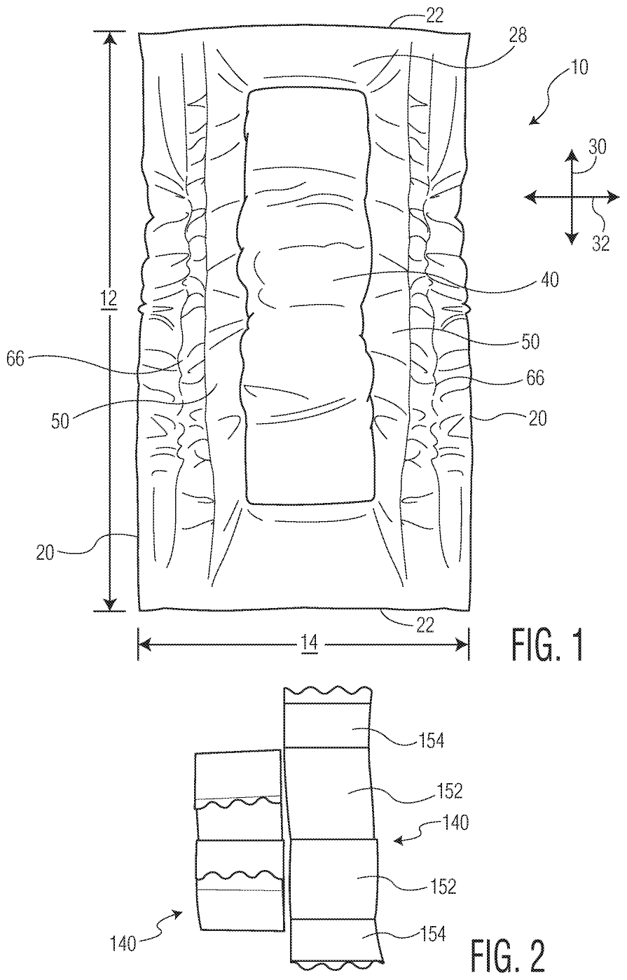

Referring to FIG. 1, a rectangular absorbent article 10 of the present disclosure is exemplified in the form of a diaper. While the embodiments and illustrations described herein may generally apply to absorbent articles manufactured in the product longitudinal direction, which is hereinafter called the machine direction manufacturing of a product, it should be noted that one of ordinary skill could apply the information herein to absorbent articles manufactured in the latitudinal direction of the product which hereinafter is called the cross direction manufacturing of a product without departing from the spirit and scope of the disclosure. The rectangular absorbent article 10 illustrated in FIG. 1 includes a length 12 in a longitudinal direction 30 and a width 14 in a lateral direction 32. Because the rectangular absorbent article 10 is intended for premature infants, it does not define waist regions (front, back and crotch) as regular absorbent articles typically do. However, the rectangular absorbent article 10 has a pair of opposed longitudinal side edges 20 and a pair of opposite waist edges 22. The length 12 of the rectangular absorbent article 10 is typically less than 310 mm. Generally speaking, for premature infants weighing 6 lbs. or less, the length 12 of the rectangular absorbent article 10 is less than 310 mm; for premature infants weighing 4 lbs. or less, the length 12 of the rectangular absorbent article 10 is equal to or less than 240 mm; and for premature infants weighing 2 lbs. or less, the length 12 of the rectangular absorbent article 10 is equal to or less than 185 mm. The width 14 of the rectangular absorbent article 10 is typically less than 200 mm. The width 14 of the rectangular absorbent article 10 may be less than 150 mm in order to provide appropriate fit and comfort for a premature infant. Rectangular absorbent articles 10 having a width 14 less than 150 mm may allow for better positioning of the premature infant and may be more appropriately sized to minimize effects of use of an absorbent article. Generally speaking, for premature infants weighing 6 lbs. or less, the width 14 of the rectangular absorbent article 10 is less than 200 mm; for premature infants weighing 4 lbs. or less, the width 14 of the rectangular absorbent article 10 is less than 140 mm; and for premature infants weighing 2 lbs. or less, the width 14 of the rectangular absorbent article 10 is less than 110 mm. The absorbent article 10 includes an outer layer 26 and a skin facing layer 28. In an embodiment, the skin facing layer 28 can be bonded to the outer layer 26 in a superposed relation by any suitable means such as, but not limited to, adhesives, ultrasonic bonds, thermal bonds, pressure bonds, or other conventional techniques. The outer layer 26 can define a length, or longitudinal direction 30, and a width, or lateral direction 32, which, in the illustrated embodiment, can coincide with the length and width of the rectangular absorbent article 10. The longitudinal direction 30 and the lateral direction 32 of the absorbent article 10, and of the materials which form the rectangular absorbent article 10, can provide the X-Y planes, respectively, of the rectangular absorbent article 10 and of the materials which form the rectangular absorbent article 10. The rectangular absorbent article 10, and the materials which form the rectangular absorbent article 10, can also have a Z-direction.

Referring to FIG. 1, an absorbent structure 40 is disposed between the outer layer 26 and the skin facing layer 28. In an embodiment, the absorbent structure 40 can have a length and width that are the same as or less than the length and width of the rectangular absorbent article 10. In an embodiment, a pair of containment flaps, each referred to by reference numeral 50, can be present and can inhibit the lateral flow of body exudates. Suitable construction and arrangements for containment flaps 50 are generally well known to those skilled in the art and are described in U.S. Pat. No. 4,704,116 issued Nov. 3, 1987, to Enloe and U.S. Pat. No. 5,562,650 issued Oct. 8, 1996 to Everett et al., which are incorporated herein by reference.

### Outer Layer:

The outer layer 26 can be breathable and/or liquid impermeable. The outer layer 26 can be elastic, stretchable or non-stretchable. The outer layer 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer layer 26 can be constructed of a microporous polymeric film, such as polyethylene or polypropylene.

In an embodiment, the outer layer 26 can be a single layer of a liquid impermeable material. In an embodiment, the outer layer 26 can be suitably stretchable, and more suitably elastic, in at least the lateral direction 32 of the rectangular absorbent article 10. In an embodiment, the outer layer 26 can be stretchable, and more suitably elastic, in both the lateral 32 and the longitudinal 30 directions. In an embodiment, the outer layer 26 can be a multi-layered laminate in which at least one of the layers is liquid impermeable. The outer layer 26 may be a two layer construction, including an outer layer material and an inner layer material which can be bonded together such as by a laminate adhesive. Suitable laminate adhesives can be applied continuously or intermittently as beads, a spray, parallel swirls, or the like. Suitable adhesives can be obtained from Bostik Findlay Adhesives, Inc. of Wauwatosa, Wis., U.S.A. It is to be understood that the inner layer can be bonded to the outer layer utilizing ultrasonic bonds, thermal bonds, pressure bonds, or the like.

The outer layer material of the outer layer 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer material of an outer layer 26 can be a 20 gsm spunbond polypropylene non-woven web.

The liquid impermeable inner layer material of the outer layer 26 (or the liquid impermeable outer layer 26 where the outer layer 26 is of a single-layer construction) can be either vapor permeable (i.e., “breathable”) or vapor impermeable. The liquid impermeable inner layer material (or the liquid impermeable outer layer 26 where the outer layer 26 is of a single-layer construction) may be manufactured from a thin plastic film, although other liquid impermeable materials may also be used. The liquid impermeable inner layer material (or the liquid impermeable outer layer 26 where the outer layer 26 is of a single-layer construction) can inhibit liquid body exudates from leaking out of the rectangular absorbent article 10 and wetting articles, such as bed sheets and clothing, as well as the wearer and caregiver. An example of a material for a liquid impermeable inner layer material (or the liquid impermeable outer layer 26 where the outer layer 26 is of a single-layer construction) can be a printed 19 gsm Berry Plastics XP-8695H film or equivalent commercially available from Berry Plastics Corporation, Evansville, Ind., U.S.A.

Where the outer layer 26 is of a single layer construction, it can be embossed and/or matte finished to provide a more cloth-like texture or appearance. The outer layer 26 can permit vapors to escape from the rectangular absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Absorbent Structure:

The absorbent structure 40 can be suitably constructed to be generally compressible, conformable, pliable, non-irritating to the wearer's skin and capable of absorbing and retaining liquid body exudates. The absorbent structure 40 can be manufactured in a wide variety of sizes and shapes (for example, rectangular, trapezoidal, T-shape, I-shape, hourglass shape, etc.) and from a wide variety of materials. The size and the absorbent capacity of the absorbent structure 40 should be compatible with the size of the intended wearer and the liquid loading imparted by the intended use of the rectangular absorbent article 10.

In an embodiment, the absorbent structure 40 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent structure 40 can be a matrix of cellulosic fluff and superabsorbent material.

In an embodiment, the absorbent structure 40 may be constructed of a single layer of materials, or in the alternative, may be constructed of two layers of materials or more. In an embodiment in which the absorbent structure 40 has two layers, the absorbent structure 40 can have a wearer facing layer suitably composed of hydrophilic fibers and an outer facing layer suitably composed at least in part of a high absorbency material commonly known as superabsorbent material. In such an embodiment, the wearer facing layer of the absorbent structure 40 can be suitably composed of cellulosic fluff, such as wood pulp fluff, and the outer facing layer of the absorbent structure 40 can be suitably composed of superabsorbent material, or a mixture of cellulosic fluff and superabsorbent material. As a result, the wearer facing layer can have a lower absorbent capacity per unit weight than the outer facing layer. The wearer facing layer may alternatively be composed of a mixture of hydrophilic fibers and superabsorbent material, as long as the concentration of superabsorbent material present in the wearer facing layer is lower than the concentration of superabsorbent material present in the outer facing layer so that the wearer facing layer can have a lower absorbent capacity per unit weight than the outer facing layer. It is also contemplated that the outer facing layer may be composed solely of superabsorbent material without departing from the scope of this disclosure. It is also contemplated that, in an embodiment, each of the layers, the wearer facing and outer facing layers, can have a superabsorbent material such that the absorbent capacities of the two superabsorbent materials can be different and can provide the absorbent structure 40 with a lower absorbent capacity in the wearer facing layer than in the outer facing layer.

Various types of wettable, hydrophilic fibers can be used in the absorbent structure 40. Examples of suitable fibers include natural fibers, cellulosic fibers, synthetic fibers composed of cellulose or cellulose derivatives, such as rayon fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In an embodiment, the cellulosic fluff can include a blend of wood pulp fluff. An example of wood pulp fluff can be “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

The absorbent structure 40 can be formed with a dry-forming technique, an air-forming technique, a wet-forming technique, a foam-forming technique, or the like, as well as combinations thereof. A coform nonwoven material may also be employed. Methods and apparatus for carrying out such techniques are well known in the art.

Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. Typically, a superabsorbent material can be capable of absorbing at least about ten times its weight in liquid. In an embodiment, the superabsorbent material can absorb more than twenty-four times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes having a largest greatest dimension/smallest dimension ratio, such as needles, flakes, and fibers are also contemplated for use herein. Conglomerates of particles of superabsorbent materials may also be used in the absorbent body structure.

In an embodiment, the absorbent structure 40 can be free of superabsorbent material. In an embodiment, the absorbent structure 40 can have at least about 15% by weight of a superabsorbent material. In an embodiment, the absorbent structure 40 can have at least about 15, 20, 25, 30, 35, 40, 45, 50, 55, 60, 65, 70, 75, 80, 85, 90, 95, 99 or 100% by weight of a superabsorbent material. In an embodiment, the absorbent structure 40 can have less than about 100, 99, 95, 90, 85, 80, 75, 70, 65, 60, 55, 50, 45, 40 35, 30, 25, or 20% by weight of a superabsorbent material. In an embodiment, the absorbent structure 40 can have from about 15, 20, 25, 30, 35, 40, 45, 50, 55 or 60% to about 65, 70, 75, 80, 85, 90, 95, 99 or 100% by weight of a superabsorbent material. Examples of superabsorbent material include, but are not limited to, FAVOR SXM-9300 or equivalent available from Evonik Industries, Greensboro, N.C., U.S.A. and HYSORB 8760 or equivalent available from BASF Corporation, Charlotte, N.C., U.S.A.

The absorbent structure 40 can be superposed over the inner layer material of the outer layer 26, extending laterally between the leg elastic members 66 and can be bonded to the inner layer material of the outer layer 26, such as by being bonded thereto with adhesive. However, it is to be understood that the absorbent structure 40 may be in contact with, and not bonded with, the outer layer 26 and remain within the scope of this disclosure. In an embodiment, the outer layer 26 can be composed of a single layer and the absorbent structure 40 can be in contact with the singer layer of the outer layer 26. In an embodiment, a layer, such as but not limited to, a fluid transfer layer (not shown in FIG. 1), can be positioned between the absorbent structure 40 and the outer layer 26.

### Fluid Transfer Lager:

In various embodiments, the rectangular absorbent article 10 can have a fluid transfer layer (not shown in the FIGS.). The fluid transfer layer can have a wearer facing surface and an outer facing surface. In an embodiment, the fluid transfer layer can be in contact with the absorbent structure 40. In an embodiment, the fluid transfer layer can be bonded to the absorbent structure 40. Bonding of the fluid transfer layer to the absorbent structure 40 can occur via any means known to one of ordinary skill, such as, but not limited to, adhesives. In an embodiment, a fluid transfer layer can be positioned between the skin facing layer 28 and the absorbent structure 40. In an embodiment, a fluid transfer layer can completely encompass the absorbent structure 40 and can be sealed to itself. In such an embodiment, the fluid transfer layer may be folded over on itself and then sealed using, for example, heat and/or pressure. In an embodiment, a fluid transfer layer may be composed of separate sheets of material which can be utilized to partially or fully encompass the absorbent structure 40 and which can be sealed together using a sealing means such as an ultrasonic bonder or other thermochemical bonding means or the use of an adhesive.

In an embodiment, the fluid transfer layer can be in contact with and/or bonded with the wearer facing surface of the absorbent structure 40. In an embodiment, the fluid transfer layer can be in contact with and/or bonded with the wearer facing surface and at least one of the edges of the absorbent structure 40. In an embodiment, the fluid transfer layer can be in contact with and/or bonded with the wearer facing surface, at least one of the edges and the outer facing surface of the absorbent structure 40. In an embodiment, the absorbent structure 40 may be partially or completely encompassed by a fluid transfer layer.

The fluid transfer layer can be pliable, less hydrophilic than the absorbent structure 40, and sufficiently porous to thereby permit liquid body exudates to penetrate through the fluid transfer layer to reach the absorbent structure 40. In an embodiment, the fluid transfer layer can have sufficient structural integrity to withstand wetting thereof and of the absorbent structure 40. In an embodiment, the fluid transfer layer can be constructed from a single layer of material or it may be a laminate constructed from two or more layers of material.

In an embodiment, the fluid transfer layer can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp. Wood pulps can include, but are not limited to, standard softwood fluffing grade such as “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

In various embodiments, the fluid transfer layer can include cellulosic material. In various embodiments, the fluid transfer layer can be creped wadding or a high-strength tissue. In various embodiments, the fluid transfer layer can include polymeric material. In an embodiment, a fluid transfer layer can include a spunbond material. In an embodiment, a fluid transfer layer can include a meltblown material. In an embodiment, the fluid transfer layer can be a laminate of a meltblown nonwoven material having fine fibers laminated to at least one spunbond nonwoven material layer having coarse fibers. In such an embodiment, the fluid transfer layer can be a spunbond-meltblown (“SM”) material. In an embodiment, the fluid transfer layer can be a spunbond-meltblown-spunbond (“SMS”) material. A non-limiting example of such a fluid transfer layer can be a 10 gsm spunbond-meltblown-spunbond material. In various embodiments, the fluid transfer layer can be composed of at least one material which has been hydraulically entangled into a nonwoven substrate. In various embodiments, the fluid transfer layer can be composed of at least two materials which have been hydraulically entangled into a nonwoven substrate. In various embodiments, the fluid transfer layer can have at least three materials which have been hydraulically entangled into a nonwoven substrate. A non-limiting example of a fluid transfer layer can be a 33 gsm hydraulically entangled substrate. In such an example, the fluid transfer layer can be a 33 gsm hydraulically entangled substrate composed of a 12 gsm spunbond material, a 10 gsm wood pulp material having a length from about 0.6 cm to about 5.5 cm, and an 11 gsm polyester staple fiber material. To manufacture the fluid transfer layer just described, the 12 gsm spunbond material can provide a base layer while the 10 gsm wood pulp material and the 11 gsm polyester staple fiber material can be homogeneously mixed together and deposited onto the spunbond material and then hydraulically entangled with the spunbond material.

In various embodiments, a wet strength agent can be included in the fluid transfer layer. A non-limiting example of a wet strength agent can be Kymene 6500 (557LK) or equivalent available from Ashland Inc. of Ashland, Ky., U.S.A. In various embodiments, a surfactant can be included in the fluid transfer layer. In various embodiments, the fluid transfer layer can be hydrophilic. In various embodiments, the fluid transfer layer can be hydrophobic and can be treated in any manner known in the art to be made hydrophilic.

In an embodiment, the fluid transfer layer can be in contact with and/or bonded with an absorbent structure 40 which is made at least partially of particulate material such as superabsorbent material. In an embodiment in which the fluid transfer layer at least partially or completely encompasses the absorbent structure 40, the fluid transfer layer should not unduly expand or stretch as this might cause the particulate material to escape from the absorbent structure 40. In an embodiment, the fluid transfer layer, while in a dry state, should have respective extension values at peak load in the machine and cross directions of 30 percent or less and 40 percent or less, respectively.

### Acquisition Layer:

In various embodiments, but not shown in the FIGURES, the rectangular absorbent article 10 can have an acquisition layer. The acquisition layer can help decelerate and diffuse surges or gushes of liquid body exudates penetrating the skin facing layer 28. In an embodiment, the acquisition layer can be positioned between the skin facing layer 28 and the absorbent structure 40 to take in and distribute body exudates for absorption by the absorbent structure 40. In an embodiment, the acquisition layer can be positioned between the skin facing layer 28 and a fluid transfer layer if a fluid transfer layer is present.

The acquisition layer can have a wearer facing surface and an outer facing surface. In an embodiment, the acquisition layer can be in contact with and/or bonded with the skin facing layer 28. In an embodiment in which the acquisition layer is bonded with the skin facing layer 28, bonding of the acquisition layer to the skin facing layer 28 can occur through the use of an adhesive and/or point fusion bonding. The point fusion bonding can be selected from, but is not limited to, ultrasonic bonding, pressure bonding, thermal bonding, and combinations thereof. In an embodiment, the point fusion bonding can be provided in any pattern as deemed suitable.

In an embodiment, the longitudinal length of the acquisition layer can be the same as the longitudinal length of the absorbent structure 40. In such an embodiment the midpoint of the longitudinal length of the acquisition layer can substantially align with the midpoint of the longitudinal length of the absorbent structure 40. In an embodiment, the longitudinal length of the acquisition layer can be shorter than the longitudinal length of the absorbent structure 40. In such an embodiment, the acquisition layer may be positioned at any desired location along the longitudinal length of the absorbent structure 40.

In an embodiment, the acquisition layer can include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. In an embodiment, the acquisition layer can be formed from a material that is substantially hydrophobic, such as a nonwoven web composed of polypropylene, polyethylene, polyester, and the like, and combinations thereof.

### Skin Facing Layer:

In various embodiments, the skin facing layer 28 of the rectangular absorbent article 10 can overlay the absorbent structure 40 and the outer layer 26 and can isolate the wearer's skin from liquid waste retained by the absorbent structure 40. In an embodiment, the skin facing layer 28 can extend beyond the absorbent structure 40 and/or a fluid transfer layer, and/or an acquisition layer to overlay a portion of the outer layer 26 and can be bonded thereto by any method deemed suitable, such as, for example, by being bonded thereto by adhesive, to substantially enclose the absorbent structure 40 between the outer layer 26 and the skin facing layer 28. The skin facing layer 28 may be narrower than the outer layer 26, but it is to be understood that the skin facing layer 28 and the outer layer 26 may be of the same dimensions. It is also contemplated that the skin facing layer 28 may not extend beyond the absorbent structure 40 and/or may not be secured to the outer layer 26. The skin facing layer 28 can be suitably compliant, soft feeling, and non-irritating to the wearer's skin and can be the same as or less hydrophilic than the absorbent structure 40 to permit body exudates to readily penetrate through to the absorbent structure 40 and provide a relatively dry surface to the wearer.

The skin facing layer 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the skin facing layer 28. The skin facing layer 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof.

For example, the skin facing layer 28 can be composed of a meltblown or spunbond web of polyolefin fibers. Alternatively, the skin facing layer 28 can be a bonded-carded web composed of natural and/or synthetic fibers. The skin facing layer 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire skin facing layer 28 or it can be selectively applied to particular sections of the skin facing layer 28. In an embodiment, the skin facing layer 28 can be treated with a modifier which can increase the surface energy of the material surface or reduce the viscoelastic properties of body exudates, such as menses.

In an embodiment, a skin facing layer 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a skin facing layer 28 can be a spunbond substrate with a basis weight from about 10 or 12 to about 15 or 20 gsm. In an embodiment, a skin facing layer 28 can be a 12 gsm spunbond-meltblown-spunbond substrate having 10% meltblown content applied between the two spunbond layers.

Although the outer layer 26 and skin facing layer 28 can include elastomeric materials, it is contemplated that the outer layer 26 and the skin facing layer 28 can be composed of materials which are generally non-elastomeric. In an embodiment, the skin facing layer 28 can be stretchable, and more suitably elastic. In an embodiment, the skin facing layer 28 can be suitably stretchable and more suitably elastic in at least the lateral direction 32 of the rectangular absorbent article 10. In other aspects, the skin facing layer 28 can be stretchable, and more suitably elastic, in both the lateral 32 and the longitudinal 30 directions.

### Containment Flaps:

In an embodiment, opposed containment flaps 50 can be secured to the skin facing layer 28 of the rectangular absorbent article 10 in a generally parallel, spaced relation with each other laterally inward of the longitudinal edges 20 to provide a barrier against the flow of body exudates. In an embodiment, the containment flaps 50 can extend longitudinally between the waist edges 22 of the rectangular absorbent article 10. The containment flaps 50 can be bonded to the skin facing layer 28 by a seam of adhesive.

The containment flaps 50 can be constructed of a fibrous material which can be similar to the material forming the skin facing layer 28. Other conventional material, such as polymer films, can also be employed. Each containment flap 50 can have a moveable distal end which can include flap elastics. Suitable elastic materials for the flap elastic can include sheets, strands or ribbons of natural rubber, synthetic rubber, or thermoplastic elastomeric materials.

The flap elastics can have two strands of elastomeric material extending longitudinally along the distal ends of the containment flaps 50, in generally parallel, spaced relation with each other. The elastic strands can be within the containment flaps 50 while in an elastically contractible condition such that contraction of the strands gathers and shortens the distal ends of the containment flaps 50. As a result, the elastic strands can bias the distal ends of each containment flap 50 toward a position spaced from the proximal end of the containment flaps 50, so that the containment flaps 50 can extend away from the skin facing layer 28 in a generally upright orientation of the containment flaps 50 when the rectangular absorbent article 10 is fitted on the wearer. The distal end of the containment flaps 50 can be connected to the flap elastics by partially doubling the containment flap 50 material back upon itself by an amount which can be sufficient to enclose the flap elastics. It is to be understood, however, that the containment flaps 50 can have any number of strands of elastomeric material and may also be omitted from the rectangular absorbent article 10 without departing from the scope of this disclosure.

### Leg Elastics:

Leg elastic members 66 can be secured between the outer and inner material layers of the outer layer 26, such as by being bonded therebetween by laminate adhesive, generally adjacent the lateral outer edges of the inner layer material of the outer layer 26. Alternatively, the leg elastic members 66 may be disposed between other layers of the rectangular absorbent article 10. A wide variety of elastic materials may be used for the leg elastic members 66. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

### Detached Fastener:

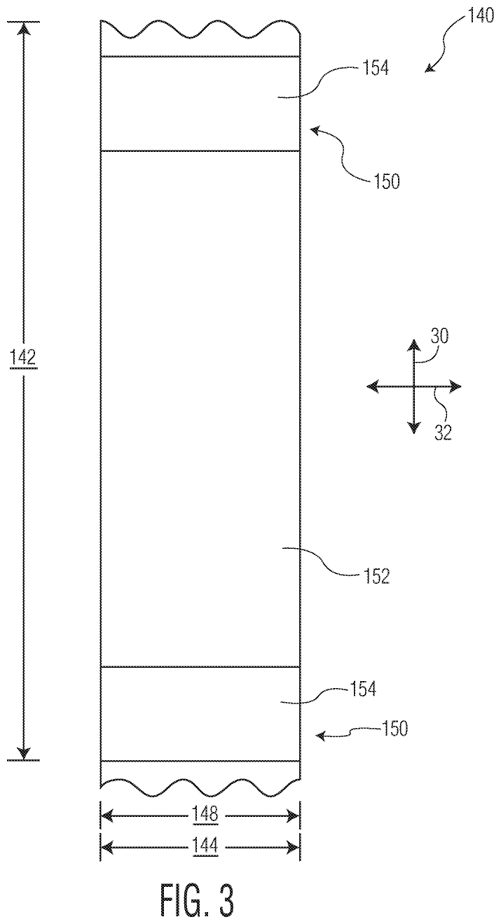

The dimensions and components of the rectangular absorbent article 10 are described herein; the system of the invention also includes a detached fastener 140. The dimensions and properties of the detached fastener 140 are selected to work in conjunction with the rectangular absorbent article 10 as described. The detached fastener 140 of the system is representatively shown in FIG. 2; the detached fastener 140 in FIG. 2 is shown in a folded configuration on the left and in an unfolded or open configuration on the right of FIG. 2. The system of the invention may include more than one detached fastener 140. The detached fastener 140 has a length 142 aligned with the lateral direction 32 of the rectangular absorbent article 10 and the detached fastener 140 has a width 144 aligned with the longitudinal direction 30 of the rectangular absorbent article 10—as can be seen in FIG. 3. The length 142 of the detached fastener 140 may be at least 50 mm. This length 142 is selected in order for the detached fastener 140 to be reasonably manipulated by a caregiver to a premature infant. Referring back to FIG. 2, the detached fastener 140 includes two, lateral end portions 150. Each of the lateral end portions 150 includes an engagement material 154. The lateral end portions 150 may be connected to each other by a nonwoven material 152. The nonwoven material 152 may be an elastic material. The nonwoven material 152 may be a spunbond material, a spunbond-meltblown-spunbond (“SMS”) material, a necked bonded laminate (“NBL”) or other suitable material. Suitable elastic materials include elastic films with nonwoven facings, such as NBL materials. The nonwoven material 152 may further be formed of stretchable materials selected from films, film laminates, spandex laminates, stretch-bonded laminate (SBL), continuous filament stretch-bonded laminates (CFSBL) and simultaneously aperture and bonded bi-directional elastic laminate (SABBEL).

The engagement material 154 may include refastenable fasteners suitable for absorbent articles, such as adhesive fasteners, cohesive fasteners, mechanical fasteners, or the like. In one suitable embodiment, the engagement material 154 includes a mechanical fastening component, such as a hook material. For example, a suitable hook material can be provided by interlocking geometric shaped materials. As used herein, “hook” broadly refers to any suitable mechanical fastener adapted to engage loop components (such “loop component” may be the outer layer 26) including, e.g., hooks, bulbs, mushrooms, arrowheads, balls on stems, stems, structures having stems that engage foam such as open cell foam or the like, etc. The shape, density, and polymer composition of the hooks may be selected to obtain the desired level of engagement between the engagement material 154 and the outer layer 26. A more aggressive hook material may comprise a material with a greater average hook height and/or a greater percentage of directionally-aligned hooks. With the systems of the invention, the engagement material 154 is selected to engage with the outward surface of the outer layer 26. The outer layer 26 may be formed of a material such as a spunbond or other suitable material.

Referring to FIG. 3, a detached fastener 140 of the system is shown in a flat, two-dimensional position. The detached fastener 140 has a length 142 that is aligned with and coincides with the lateral direction 32 of the rectangular absorbent article 10 and the detached fastener 140 has a width 144 that is aligned with and coincides with the longitudinal direction 30 of the rectangular absorbent article 10. The detached fastener 140 includes two, opposed lateral end portions 150. Each lateral end portion includes an engagement material 154. The engagement material 154 defines a length 146 that is oriented with the length 142 of the detached fastener 140 and the engagement material 154 defines a width 148 that is oriented with the width 144 of the detached fastener 140. In order to function with the rectangular absorbent articles 10 of the system of the invention, the dimensions (length 146 and width 148) of the engagement material 154 are selected for optimal use with the smaller dimensions of the rectangular absorbent article 10. The length 146 of the engagement material 154 may be less than 35 mm. The width 148 of the engagement material 154 may be less than 45 mm. More particularly, for a rectangular absorbent article 10 intended for use with premature infants weighing 6 lbs. or less, the length 146 of the engagement material 154 may be about 20 mm and the width 148 of the engagement material 154 may be about 25 mm. For a rectangular absorbent article 10 intended for use with premature infants weighing 4 lbs. or less, the length 146 of the engagement material 154 may be about 15 mm and the width 148 of the engagement material 154 may be from 20 to 30 mm. Further, for a rectangular absorbent article 10 intended for use with premature infants weighing 2 lbs. or less, the length 146 of the engagement material 154 may be about 10 mm and the width 148 of the engagement material 154 may be about 15 mm. The length 142 of the detached fastener 140 is desirably at least 50 mm and may be 60 mm, 75 mm or 90 mm.

The relationship between the length 12 of the rectangular absorbent article 10 and the width 148 of the engagement material 154 of the detached fastener 140 may be selected based on a rational relationship between the two dimensions. For example, the system of the invention may have a ratio of the length 12 of the rectangular absorbent article 10 to the width 148 of the engagement material 154 of detached fastener 140 of at least 20 to 3. An example of execution of this ratio is a rectangular absorbent article 10 having a length 12 of 300 mm and an engagement material 154 on the detached fastener 140 having a width 148 of 45 mm. With the smaller sizes of the rectangular absorbent articles 10, engagement materials 154 having a width 148 greater than 45 mm may touch the thigh of the premature infant; this could create friction against the skin. Therefore, the width 148 of the engagement material 154 is desirably equal to or less than 45 mm.

In another embodiment, the system of the invention may have a ratio of the length 12 of the rectangular absorbent article 10 to the width 148 of the engagement material 154 of the detached fastener 140 of less than 40 to 3. An example of the execution of this ratio is a rectangular absorbent article 10 having a length 12 of 180 mm and an engagement material 154 on the detached fastener 140 having a width 148 of 15 mm. This represents a ratio of 36 to 3 (or 12 to 1).

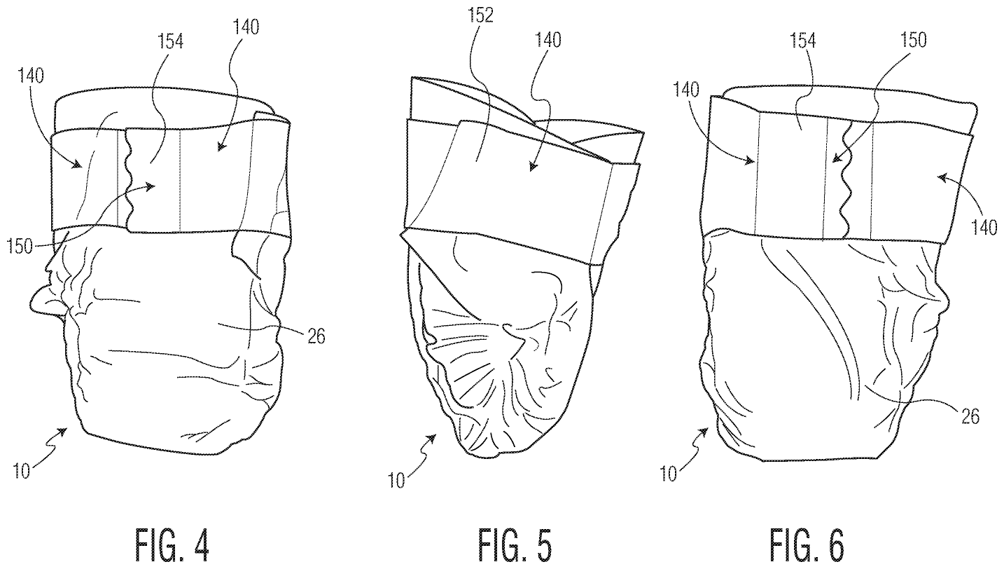

As described herein, the system of the invention may include more than one detached fastener 140 for securing the rectangular absorbent article 10 around a premature infant. FIGS. 4-6 representatively illustrate how two detached fasteners 140 can work together to secure the rectangular absorbent article around the torso of an infant. FIG. 4 shows a front view of a rectangular absorbent article 10 with the lateral end portion 150 of one detached fastener 140 visible and covering the lateral end portion 150 of a second detached fastener. The engagement material 154 would be facing toward and engaging the outer layer 26 and/or the detached fastener 140. FIG. 5 shows a side view of the same rectangular absorbent article 10 shown in FIG. 4. In the side view of FIG. 5, the nonwoven material 152 sections of the detached fasteners 140 are shown overlapping each other. FIG. 6 shows a rear view of the rectangular absorbent article shown in FIGS. 4 and 5. The rear view of FIG. 6 is similar to the front view of FIG. 4 in that a lateral end portion 150 of one of the detached fasteners 140 can be seen overlapping and engaging with the outer layer 26 and the other detached fastener 140. The engagement material 154 is facing toward and engaging the outer layer 26 and/or the detached fastener 140. While FIGS. 4-6 depict one way the elements of the system of the invention may be used together, another benefit of the system of the invention and the system element of a detached fastener 140 is a customized use/application depending on the needs of a particular infant. In addition to providing fastening around the infant's waist, the detached fasteners 140 may also be positioned and applied to the rectangular absorbent article 10 in locations that facilitate delivery of unique health and comfort needs. This benefit cannot be achieved with traditional absorbent articles having fixed fasteners. For example, the detached fastener 140 may be positioned downward toward the crotch region between the infant's legs; this positioning could help avoid the sensitive, lower abdomen (bowel) areas on the infant's torso. In another example, the detached fastener 140 may be positioned on the side or on the side/back of the rectangular absorbent article 10 to secure medical lines 156 in order to help prevent the infant pulling on the medical lines 156 or getting tangled in them when moving or abruptly stretching.

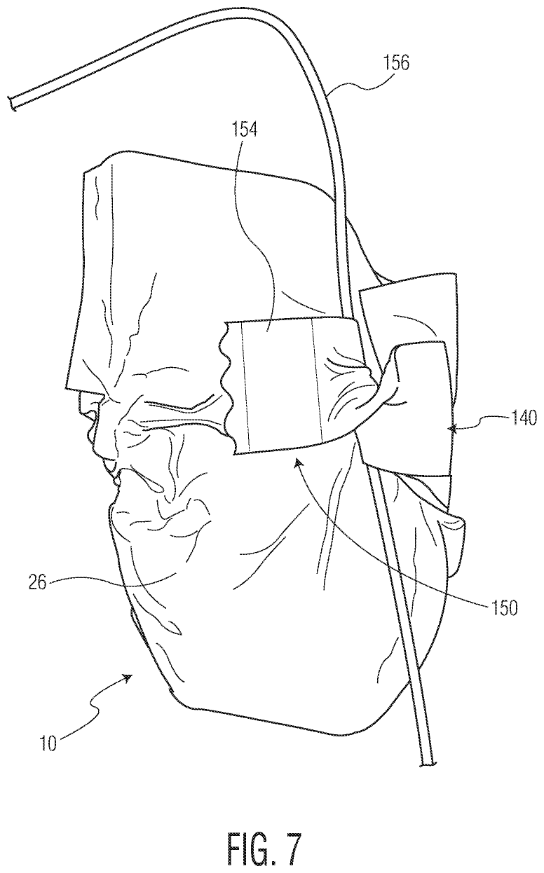

FIG. 7 representatively shows how the system of the invention can be used to secure and protect medical lines 156 being used for the care of premature infants (or other infants being treated in a NICU of a hospital). In FIG. 7, the rectangular absorbent article 10 is shown in the configuration in which it would appear if being worn by an infant. The detached fastener 140 is deployed in such a way so as to secure a medical line 156 against the outer layer 26 of the rectangular absorbent article 10. The detached fastener 140 is twisted around the medical line 156 and then the engagement material 154 of the lateral end portions 150 is secured to the outer layer 26. The systems of the invention have the structure and flexibility of use to provide improved care to small infants because they provide the traditional function of absorbing bodily exudates and keeping the exudates away from skin and open and wound areas while also providing a means to adjustably apply the rectangular absorbent article 10 and to secure the position of medical items (such as medical line 156) in the infant's care environment.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system for providing skin protection for premature infants, the system comprising:
a) a rectangular absorbent article having a longitudinal direction and a lateral direction and having a length in the longitudinal direction and a width in the lateral direction; the rectangular absorbent article includes an outer layer, an inner, skin facing layer and an absorbent structure located between the outer layer and the inner, skin facing layer, wherein the length of the rectangular absorbent article is less than 310 mm and the width of the rectangular absorbent article is less than 200 mm; and
b) a detached fastener having a length aligned with the lateral direction of the rectangular absorbent article, a width aligned with the longitudinal direction of the rectangular absorbent article and two, lateral end portions; each of the lateral end portions including an engagement material and defining a length and a width oriented with the length and the width of the detached fastener, wherein the length of the engagement material is less than 35 mm and the width of the engagement material is less than 45 mm.

2. The system of claim 1, wherein the length of the detached fastener is at least 50 mm.

3. The system of claim 1 further including more than one detached fastener.

4. The system of claim 1, wherein the end portions of the detached fastener are connected with each other by a nonwoven material.

5. The system of claim 3, wherein the nonwoven material is an elastic material.

6. The system of claim 1, wherein the engagement material of the end portions is a hook material.

7. The system of claim 1, wherein the width of the rectangular absorbent article is less than 150 mm.

8. A system for providing skin protection for premature infants, the system comprising:
a) a rectangular absorbent article having a longitudinal direction and a lateral direction and having a length in the longitudinal direction and a width in the lateral direction; the rectangular absorbent article includes an outer layer, an inner, skin facing layer and an absorbent structure located between the outer layer and the inner, skin facing layer; and
b) a detached fastener having a length aligned with the lateral direction of the rectangular absorbent article, a width aligned with the longitudinal direction of the rectangular absorbent article and two, lateral end portions; each of the lateral end portions including an engagement material and defining a length and a width oriented with the length and the width of the detached fastener, wherein a ratio of the length of the rectangular absorbent article to the width of the engagement material of the detached fastener is at least 20 to 3.

9. The system of claim 8, wherein the width of the engagement material of the detached fastener is less than 45 mm.

10. The system of claim 8, wherein the length of the rectangular absorbent article is at least 180 mm.

11. The system of claim 8, wherein the ratio of the length of the rectangular absorbent article to the width of the engagement material of the detached fastener is less than 40 to 3.

12. The system of claim 8 further including more than one detached fastener.

13. The system of claim 8, wherein the end portions of the detached fastener are connected with each other by a nonwoven material.

14. The system of claim 13, wherein the nonwoven material is an elastic material.

15. The system of claim 8, wherein the engagement material of the end portions is a hook material.

16. The system of claim 8, wherein the width of the rectangular absorbent article is less than 150 mm.
